(19) United States
(12) Patent Application Publication
(10) Pub. No.: US 2025/0148598 A1
(43) Pub. Date: May 8, 2025

(54) IMAGE INFORMATION PROCESSING APPARATUS, IMAGE INFORMATION PROCESSING METHOD, AND NON-TRANSITORY COMPUTER READABLE MEDIUM

(71) Applicants: Rigshospitalet, Copenhagen (DK); Canon Kabushiki Kaisha, Tokyo (JP); CANON MEDICAL SYSTEMS CORPORATION, Tochigi (JP)

(72) Inventors: Klaus FUGLSANG KOFOED, Copenhagen (DK); Michael Huy Cuong PHAM, Copenhagen (DK); Yukiteru MASUDA, Kawasaki (JP); Ryo ISHIKAWA, Kawasaki (JP); Gakuto AOYAMA, Kawasaki (JP)

(73) Assignees: Rigshospitalet, Copenhagen (DK); Canon Kabushiki Kaisha, Tokyo (JP); CANON MEDICAL SYSTEMS CORPORATION, Tochigi (JP)

(21) Appl. No.: 18/938,340

(22) Filed: Nov. 6, 2024

(30) Foreign Application Priority Data
Nov. 6, 2023 (JP) 2023-189632

(51) Int. Cl. G06T 7/00; G06T 5/50; G06T 7/50; G06V 10/44; G16H 30/20; G16H 30/40

(52) U.S. Cl. CPC G06T 7/0012; G06T 5/50; G06T 7/50; G06V 10/44; G16H 30/20; G16H 30/40; G06T 2207/20221; G06T 2207/30048

(57) ABSTRACT

An image information processing apparatus provided in one aspect of the present invention includes processing circuitry. The processing circuitry acquires the same structure from a plurality of medical images captured at different times or by different devices, calculates feature quantities related to the form of the structure for each of the medical images, and corrects the structure so that a difference in the feature quantities is reduced.

### CROSS-REFERENCE TO RELATED APPLICATIONS

This application is based upon and claims the benefit of priority from Japanese Patent Application No. 2023-189632, filed on Nov. 6, 2023; the entire contents of which are incorporated herein by reference.

### FIELD

Embodiments disclosed herein relate generally to an image information processing apparatus, an image information processing method, and a non-transitory computer readable medium.

### BACKGROUND

When a treatment by using medical images is planned, a region of a predetermined anatomical structure (i.e. an organ) is extracted from the medical image and various measurement values are calculated from the region. For example, the region of an organ can be extracted from a medical image by machine learning or the like, and various measurement values can be calculated from the extracted region.

However, in an organ segmentation process using machine learning or the like in the related art, even when, for example, an organ has anatomical feature quantities that are invariant over time with respect to the shape, since features related to invariance thereof are not fully taken into account, results that are inconsistent with the temporal invariance of the anatomical feature quantities may be obtained.

### BRIEF DESCRIPTION OF THE DRAWINGS

FIG. 1 is a diagram illustrating an example of the configuration of an X-ray CT apparatus including an image information processing apparatus according to an embodiment;

FIG. 2 is a flowchart illustrating the flow of a process performed by an image information processing apparatus according to a first embodiment;

FIG. 3 is a diagram illustrating an example of the extraction of a target a structure of interest in the image information processing apparatus according to the first embodiment;

FIG. 4 is a diagram illustrating the setting of correction locations in the image information processing apparatus according to the first embodiment;

FIG. 5 is a diagram illustrating the setting of correction locations in the image information processing apparatus according to the first embodiment;

FIG. 6 is a diagram explaining a correction process in the image information processing apparatus according to the first embodiment;

FIG. 7 is a diagram explaining a correction process in the image information processing apparatus according to the first embodiment;

FIG. 8 is a diagram explaining a correction process in the image information processing apparatus according to the first embodiment; and

FIG. 9 is a diagram explaining examples of GUIs according to a second variation and a third variation of the first embodiment.

### DETAILED DESCRIPTION

An image information processing apparatus provided in one aspect of the present invention includes processing circuitry. The processing circuitry acquires the same structure from a plurality of medical images taken at different times or by different devices, calculates feature quantities related to the form of the structure for each of the medical images, and corrects the structure so that a difference in the feature quantities is reduced.

Embodiments of an image information processing apparatus, an image information processing method, and a computer program are described in detail below with reference to the drawings.

### First Embodiment

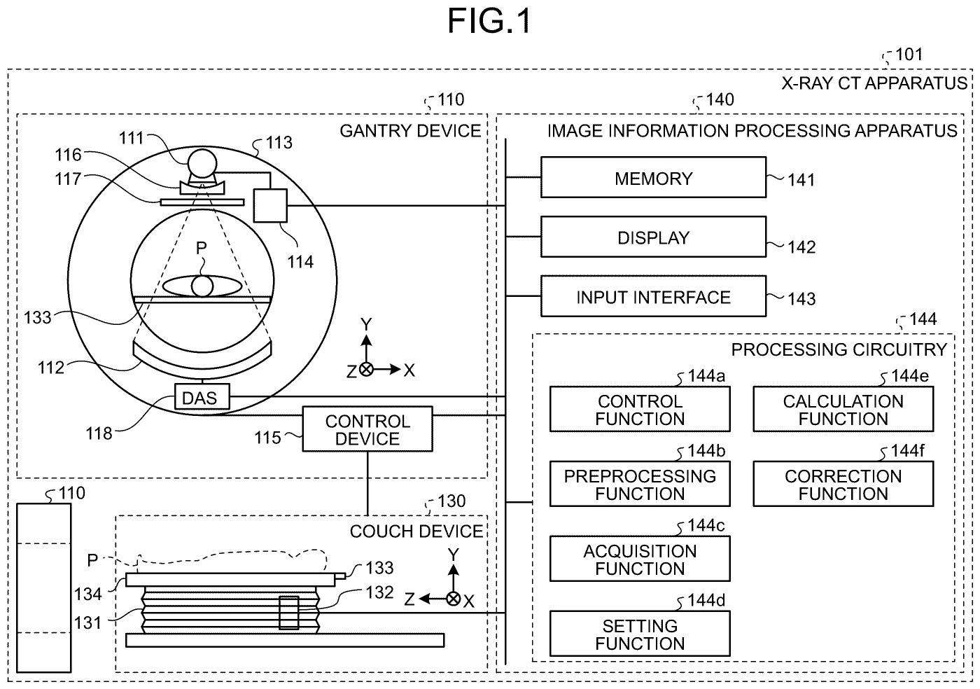

FIG. 1 illustrates an example of the configuration of an image information processing apparatus 140 according to an embodiment. In FIG. 1, a case in which the image information processing apparatus 140 according to the embodiment is a part of an X-ray CT apparatus 101 is described. The image information processing apparatus 140 is not limited to being a part of the X-ray CT apparatus 101, but may also be a part of a medical image diagnostic apparatus other than the X-ray CT apparatus such as an ultrasound diagnostic apparatus or a magnetic resonance imaging apparatus. As another example, the image information processing apparatus 140 may be configured independently of these medical image diagnostic apparatuses.

As illustrated in FIG. 1, the X-ray CT apparatus 101 according to the embodiment has a gantry device 110, a couch device 130, and the image information processing apparatus 140. FIG. 1 illustrates the gantry device 110 drawn from a plurality of directions for the purpose of explanation, and illustrates a case in which the X-ray CT apparatus 101 has one gantry device 110.

The gantry device 110 has an X-ray tube 111, an X-ray detector 112, a rotating frame 113, an X-ray high voltage device 114, a control device 115, a wedge 116, a collimator 117, and a data acquisition system (DAS) 118.

The X-ray tube 111 is a vacuum tube with a cathode (filament) that generates hot electrons and an anode (target) that generates X-rays upon impact of the hot electrons. The X-ray tube 111 generates X-rays that are emitted to a subject P by hot electrons from the cathode to the anode due to the application of high voltage from the X-ray high voltage device 114. For example, the X-ray tube 111 is a rotating anode X-ray tube that generates X-rays by emitting hot electrons to a rotating anode.

The X-ray tube 111 and the control device 115 are an example of an x-ray irradiation unit. The x-ray irradiation unit performs a low-flux scan on a phantom made of known material and having known transmission length. Specifically, the x-ray irradiation unit performs a low-flux scan by performing an air scan and a scan on a phantom made of a plurality of different materials at the initial current intensity and respective tube voltage settings of the X-ray tube.

The rotating frame 113 is an annular frame that supports the X-ray tube 111 and the X-ray detector 112 opposite each other and rotates the X-ray tube 111 and the X-ray detector 112 by using the control device 115. For example, the rotating frame 113 is a casting made of aluminum. In addition to the X-ray tube 111 and the X-ray detector 112, the rotating frame 113 can further support the X-ray high voltage device 114, the wedge 116, the collimator 117, the DAS 118, and the like. Moreover, the rotating frame 113 can further support various configurations not illustrated in FIG. 1.

The wedge 116 is a filter used to regulate the x-ray dose emitted from the X-ray tube 111. Specifically, the wedge 116 is a filter that transmits and attenuates X-rays emitted from the X-ray tube 111 so that the distribution of the X-rays emitted from the X-ray tube 111 to the subject P is a predetermined distribution. For example, the wedge 116 is a wedge filter or a bow-tie filter, and is a filter made of aluminum or other material processed to have a predetermined target angle and a predetermined thickness.

The collimator 117 is a lead plate or the like for narrowing the irradiation range of X-rays transmitted through the wedge 116, and forms a slit by combining a plurality of lead plates or the like. The collimator 117 may also be referred to as an x-ray diaphragm. FIG. 1 illustrates a case in which the wedge 116 is placed between the X-ray tube 111 and the collimator 117; however, the collimator 117 may also be placed between the X-ray tube 111 and the wedge 116. In this case, the wedge 116 attenuates X-rays emitted from the X-ray tube 111 and having an irradiation range limited by the collimator 117 by allowing the X-rays to pass therethrough.

The X-ray high voltage device 114 has electrical circuitry such as a transformer and a rectifier, a high voltage generator that generates a high voltage to be applied to the X-ray tube 111, and an X-ray controller that controls an output voltage corresponding to X-rays generated by the X-ray tube 111. The high voltage generator may be of a transformer type or an inverter type. The X-ray high voltage device 114 may be installed in the rotating frame 113 or in a fixed frame (not illustrated).

The control device 115 includes processing circuitry having a central processing unit (CPU) or the like, and a drive mechanism such as a motor and an actuator. The control device 115 receives input signals from an input interface 143 and controls the operations of the gantry device 110 and the couch device 130. For example, the control device 115 controls the rotation of the rotating frame 113, the tilt of the gantry device 110, the operations of the couch device 130 and a couchtop 133, and the like. The control device 115 may be installed in the gantry device 110 or in the image information processing apparatus 140.

The X-ray detector 112 is, for example, a photon-counting or energy-integrating detector. When the X-ray detector 112 is a photon-detecting detector, the X-ray detector 112 outputs a signal upon receiving an X-ray photon being a photon originating from X-rays emitted from the X-ray tube 111 and transmitted through the subject P, the signal being able to measure an energy value of the X-ray photon. The X-ray detector 112 has a plurality of X-ray detector elements that output one pulse of electrical signal (analog signal) upon receiving the X-ray photon.

The X-ray detector element is, for example, a semiconductor element (semiconductor detector element), such as cadmium telluride (CdTe) or cadmium zinc telluride (CdZnTe), in which anode and cathode electrodes are arranged.

The X-ray detector 112 has a plurality of X-ray detector elements and a plurality of application specific integrated circuits (ASIC) each being a readout circuit connected to the X-ray detector element to count an X-ray photon detected by the X-ray detector element. The ASIC counts the number of X-ray photons incident on the detector element by discriminating individual charges output by the X-ray detector element. The ASIC also measures the energy of the counted X-ray photons by performing arithmetic processing based on the magnitude of the individual charges. Moreover, the ASIC outputs the results of counting the X-ray photons to the DAS 118 as digital data.

The DAS 118 generates detection data on the basis of the results of the counting process input from the X-ray detector 112. The detection data is, for example, a sinogram. The sinogram is data that lines up the results of the counting process incident on each x-ray detector element at each position of the X-ray tube 111. The sinogram is data in which the results of the counting process are arranged in a two-dimensional orthogonal coordinate system with axes in view and channel directions. The DAS 118 generates sinograms, for example, on a per-slice column basis in the slice direction in the X-ray detector 112. The DAS 118 forwards the generated detection data to the image information processing apparatus 140. The DAS 118 is implemented with, for example, a processor.

The data generated by the DAS 118 is transmitted by optical communication from a transmitter with a light emitting diode (LED) installed in the rotating frame 113 to a receiver with a photodiode (not illustrated in FIG. 1) installed in a non-rotating part (for example, a fixed frame or the like) of the gantry device 110, and the data is then forwarded to the image information processing apparatus 140. The non-rotating part is, for example, a fixed frame that rotatably supports the rotating frame 113. The method of transmitting data from the rotating frame 113 to the non-rotating part of the gantry device 110 is not limited to optical communication; however, any non-contact data transmission method may be used or a contact data transmission method may be used.

The couch device 130 is a device for placing and moving the subject P to be photographed, and has a base 131, a couch drive device 132, the couchtop 133, and a support frame 134. The base 131 is a housing that supports the support frame 134 in a vertically movable manner. The couch drive device 132 is a drive mechanism that moves the couchtop 133, on which the subject P is placed, in the direction of a long axis of the couchtop 133, and includes a motor, an actuator, and the like. The couchtop 133 on a top surface of the support frame 134 is a board on which the subject P is placed. In addition to the couchtop 133, the couch drive device 132 may move the support frame 134 in the direction of the long axis of the couchtop 133.

The image information processing apparatus 140 has a memory 141, a display 142, the input interface 143, and processing circuitry 144. The image information processing apparatus 140 is described as a separate entity from the gantry device 110; however, the gantry device 110 may include some of the components of the image information processing apparatus 140.

The memory 141 is implemented with a semiconductor memory element such as a random access memory (RAM) and a flash memory, a hard disk, an optical disk, or the like. The memory 141 stores, for example, projection data and CT image data. For example, the memory 141 stores computer programs for the circuitry included in the X-ray CT apparatus 101 to implement various functions thereof. The memory 141 may be implemented with a server group (cloud) connected to the X-ray CT apparatus 101 via a network.

The display 142 displays various information. For example, the display 142 displays various images generated by the processing circuitry 144 or displays a graphical user interface (GUI) for receiving various operations from an operator. For example, the display 142 is a liquid crystal display or a cathode ray tube (CRT) display. The display 142 may be of a desktop type, or may be configured as a tablet terminal or the like capable of wirelessly communicating with a body of the image information processing apparatus 140. The display 142 is an example of a display unit.

The input interface 143 receives various input operations from the operator, converts the received input operations into electrical signals, and outputs the electrical signals to the processing circuitry 144. For example, the input interface 143 receives, from the operator, input operations such as scan conditions, reconstruction conditions for reconstructing CT image data, and image processing conditions for generating post-processed images from the CT image data.

For example, the input interface 143 is implemented with a mouse, a keyboard, a trackball, a switch, a button, a joystick, a touchpad for performing input operations by touching an operation surface, a touchscreen that integrates a display screen and a touchpad, non-contact input circuitry using an optical sensor, voice input circuitry, and the like. The input interface 143 may be provided on the gantry device 110. The input interface 143 may be configured as a tablet terminal or the like capable of wirelessly communicating with the body of the image information processing apparatus 140. The input interface 143 is not limited only to those with physical operating components such as a mouse and a keyboard. For example, an example of the input interface 143 also includes electrical signal processing circuitry that receives electrical signals corresponding to input operations from an external input device provided separately from the image information processing apparatus 140 and outputs the electrical signals to the processing circuitry 144.

The processing circuitry 144 controls the operation of the entire X-ray CT apparatus 101. For example, the processing circuitry 144 performs a control function 144a, a preprocessing function 144b, an acquisition function 144c, a setting function 144d, a calculation function 144e, and a correction function 144f. For example, each processing function performed by the control function 144a, the preprocessing function 144b, the acquisition function 144c, the setting function 144d, the calculation function 144e, and the correction function 144f, which are components of the processing circuitry 144 illustrated in FIG. 1, is recorded in the memory 141 in the form of a computer program executable by a computer. The processing circuitry 144 is, for example, a processor, and reads the computer programs from the memory 141 and executes the read computer programs, thereby implementing functions corresponding to the executed computer programs. In other words, the processing circuitry 144 in the state of reading the computer programs has each of the functions illustrated in the processing circuitry 144 in FIG. 1.

The control function 144a, the preprocessing function 144b, the acquisition function 144c, the setting function 144d, the calculation function 144e, and the correction function 144f are examples of a control unit, a preprocessing unit, an acquisition unit, a setting unit, a calculation unit, and a correction unit, respectively. The control unit is also an example of a display control unit. The memory 141 is an example of a storage unit.

FIG. 1 illustrates a case in which each processing function of the control function 144a, the preprocessing function 144b, the acquisition function 144c, the setting function 144d, the calculation function 144e, and the correction function 144f is implemented with single processing circuitry 144; however, the embodiment is not limited thereto. For example, the processing circuitry 144 may be configured by combining a plurality of independent processors, and respective processors may implement respective processing functions by executing respective computer programs. The processing functions of the processing circuitry 144 may be implemented by being appropriately distributed or integrated into single processing circuitry or a plurality of pieces of processing circuitry.

The control function 144a controls various processes on the basis of input operations received from the operator via the input interface 143. Specifically, the control function 144a controls CT scans performed on the gantry device 110. For example, the control function 144a controls a collection process of counting results on the gantry device 110 by controlling the operations of the X-ray high voltage device 114, the X-ray detector 112, the control device 115, the DAS 118, and the couch drive device 132. In one example, the control function 144a controls a collection process of projection data in a positioning scan for collecting positioning images (scout images) and in the imaging (main scan) for collecting images used for diagnosis.

In addition, the control function 144a, as a display control unit, causes the display 142 to display images or the like based on various image data stored in the memory 141.

The preprocessing function 144b generates projection data by performing preprocessing, such as logarithmic transformation processing, offset correction processing, inter-channel sensitivity correction processing, beam hardening correction, scattered ray correction, and dark count correction, on the detection data output from the DAS 118.

The processing circuitry 144 acquires various data from the X-ray detector 112 by using the acquisition function 144c. Details of the setting function 144d, the calculation function 144e, and the correction function 144f are described below.

The image information processing apparatus according to the embodiment includes an acquisition unit, a calculation unit, and a correction unit. The acquisition unit acquires the same structure from a plurality of medical images captured at different times or by different devices. The calculation unit calculates feature quantities related to the form of the structure for each of the medical images. The correction unit corrects the structure so that the difference in the feature quantities is reduced.

The image information processing method according to the embodiment acquires the same structure from a plurality of medical images captured at different times or by different devices, calculates feature quantities related to the form of the structure for each of the medical images, and corrects the structure so that a difference in the feature quantities is reduced.

A computer program according to the embodiment also causes a computer to perform a process of acquiring the same structure from a plurality of medical images captured at different times or by different devices, calculating feature quantities related to the form of the structure for each of the medical images, and correcting the structure so that a difference in the feature quantities is reduced.

That is, the image information processing apparatus according to the embodiment performs a correction process so that, for example, the different in feature quantities at anatomically corresponding positions at different times is reduced. The feature quantity is, for example, the length of anatomical structure. This allows the correction process of time-series images to be performed while keeping anatomical feature quantities in a reasonable form.

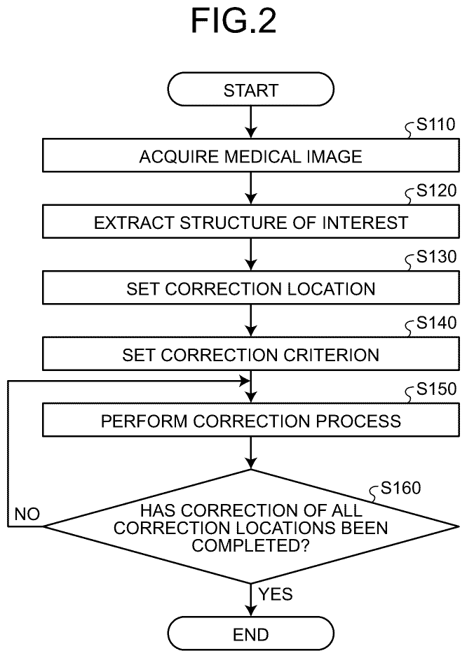

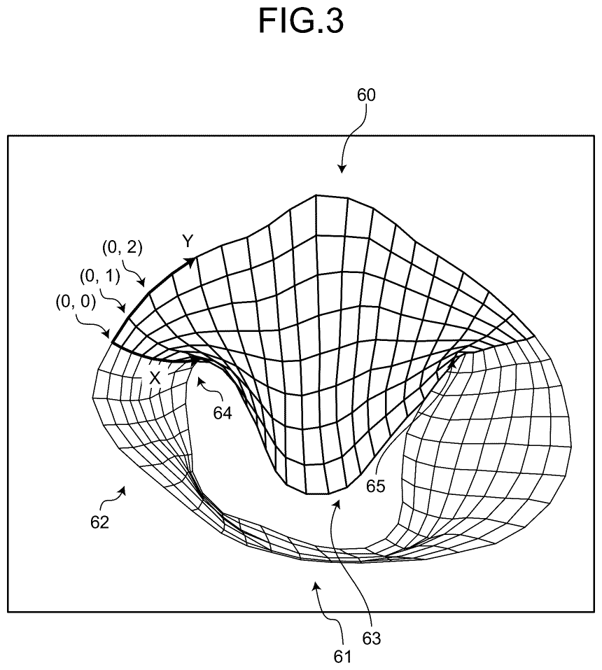

A process performed by the image information processing apparatus 140 according to the embodiment is described below with reference to FIG. 2 while referring to FIGS. 3 to 8 as appropriate. Although the following embodiments describe the case of mitral valve processing, the embodiments are not limited thereto and may be applied to other types of anatomical structures.

First, at step S110, the processing circuitry 144 acquires, by using the acquisition function 144c, an X-ray CT image of a subject as a medical image from the X-ray CT apparatus 101 or an in-hospital image database connected to the image information processing apparatus 140. As an example, the processing circuitry 144 acquires, by using the acquisition function 144c, a plurality of medical images captured at different times or by different devices.

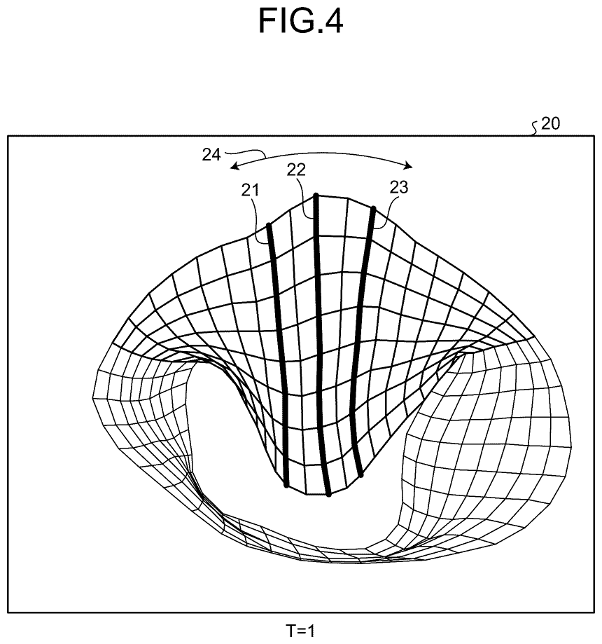

As an example, the processing circuitry 144 acquires, by using the acquisition function 144c, a plurality of CT images captured at different cardiac phases T=1, T=2, and T=3, as the medical images. As an example, the processing circuitry 144 acquires, by using the acquisition function 144c, a CT image 20 captured at the cardiac phase T=1, as illustrated in FIG. 4 to be described below.

The processing circuitry 144 acquires, by using the acquisition function 144c, the CT image 20 captured at the cardiac phase T=1 as one of the medical images, as illustrated in FIG. 4 to be described below.

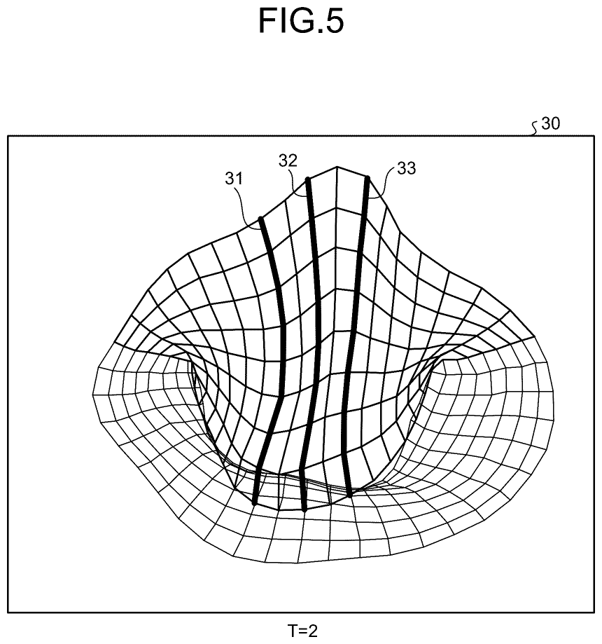

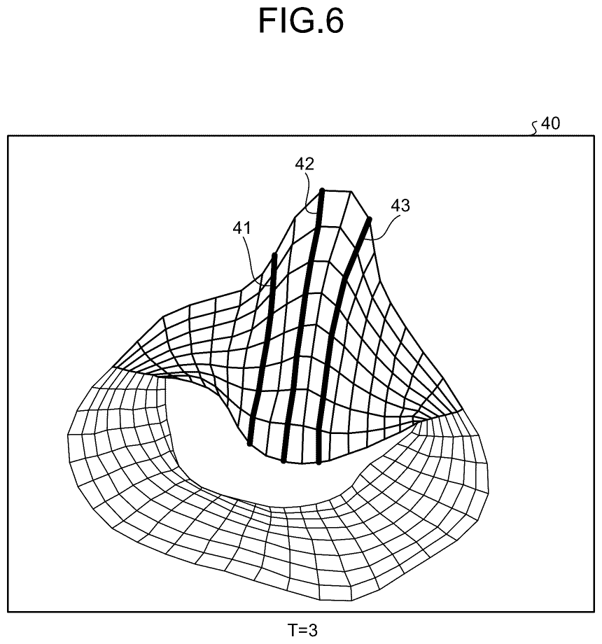

The processing circuitry 144 acquires, by using the acquisition function 144c, a CT image 30 captured at the cardiac phase T=2 as one of the medical images, as illustrated in FIG. 5 to be described below. The processing circuitry 144 acquires, by using the acquisition function 144c, a CT image 40 captured at the cardiac phase T=3 as one of the medical images, as illustrated in FIG. 6 to be described below. That is, the processing circuitry 144 acquires a 4DCT image by using the acquisition function 144c.

The degree of difference in the imaging times of the CT images acquired by the processing circuitry 144 using the acquisition function 144c is arbitrary, and may differ only for a short period of time of less than one second, for example, such as the image of each cardiac phase in the 4DCT image, or conversely, may differ for a long period of time of about one year in the imaging time, for example. The processing circuitry 144 may also acquire images as a plurality of medical images by imaging with a plurality of modalities, for example, images captured using an ultrasound diagnostic apparatus and images captured using a magnetic resonance imaging apparatus.

As an example of a condition for starting step S110, the processing circuitry 144 may acquire a CT image of the subject as the medical image by using the acquisition function 144c upon receiving an instruction from a user as a trigger. As another example, the processing circuitry 144 or another processing circuitry may monitor a medical image storage device such as a PACS, and when the processing circuitry 144 or the other processing circuitry detects that a new image has been stored in that medical image storage device, the processing circuitry 144 may start the process of step S110.

As another example, the processing circuitry 144 may determine whether the new image satisfies predetermined conditions, and start the process of step S110 when the new image satisfies the predetermined conditions. Examples of such predetermined conditions include conditions related to an imaging protocol such as a condition that the new image was captured with an imaging protocol targeting the heart, and conditions related to a reconstruction method such as a condition that the new image is an enlarged and reconstructed image.

As already described above, the medical image acquired by the processing circuitry 144 at step S110 is not limited to an X-ray CT image, but may be any other type of image in which structural information on the three-dimensional anatomical structure of a target biological tissue is stored. As an example, the medical image acquired by the processing circuitry 144 at step S110 may be an ultrasound image, an MRI image, an X-ray image, a PET image, or a SPECT image.

Subsequently, at step S120, the processing circuitry 144 extracts a structure of interest by using the acquisition function 144c. That is, the processing circuitry 144 acquires, by using the acquisition function 144c, a predetermined structure from each of the medical images acquired at step S110 and taken at different times or by different devices. For example, the processing circuitry 144 acquires the shape information of anatomical structures in the medical image as a predetermined structure by using the acquisition function 144c. That is, the processing circuitry 144 extracts, by using the acquisition function 144c, a region indicating the target anatomical structure as the predetermined structure from the CT image acquired at step S110. For example, the processing circuitry 144 extracts, by using the acquisition function 144c, a region of a mitral valve and acquires the coordinate information of pixels in the region of the mitral valve. At step S120, the processing circuitry 144 may extract, by using the acquisition function 144c, the predetermined structure by receiving manual designation of the position of the structure of interest using the user interface, or may automatically extract the predetermined structure by using known region extraction techniques. Examples of such region extraction techniques include the Otsu binarization method based on CT values, the region expansion method, the snake method, the graph cut method, and the Mean Shift method.

The example of extracting the predetermined structure at step S120 is not limited to the method described above, and the processing circuitry 144 may acquire, by using the acquisition function 144c, the predetermined structure by extracting the predetermined structure from a shape model constructed by learning data prepared in advance using machine learning technology including deep learning.

As another example, the processing circuitry 144 may extract, by using the acquisition function 144c, a related region being a region larger than the region of the target anatomical structure but smaller than the region of the entire image acquired at step S110, and use the above-described method for the related region. This can reduce the computational cost required at step S120. As an example, when the mitral valve is acquired as the predetermined structure, the processing circuitry 144 extracts, by using the acquisition function 144c, a heart region as a related region and acquires the predetermined structure from the related region by using known region extraction techniques or the like. As another example, the processing circuitry 144 may extract, by using the acquisition function 144c, the region of the sum set of the region of the left atrium and the region of the right atrium, for example, as a related region, and acquire the predetermined structure from the related region by using known region extraction techniques or the like. In setting the related region, the processing circuitry 144 may set, by using the acquisition function 144c, the related region by receiving input from a user through the user interface.

The region indicating the target anatomical structure may be identified separately for each region with different features and characteristics within that region. As an example, in the case of extracting the mitral valve, since the mitral valve is constituted by two valve leaflets of the anterior leaflet and the posterior leaflet, the processing circuitry 144 may acquire, by using the acquisition function 144c, the predetermined structure by using the anterior leaflet region and the posterior leaflet region as a plurality of regions and using known region extraction techniques or the like for each of the regions.

FIG. 3 illustrates an example of the predetermined structure of the mitral valve region at a certain time extracted at step S120 by the processing circuitry 144 using the acquisition function 144c. In FIG. 3, an arrow 60 indicates the anterior leaflet and an arrow 61 indicates the posterior leaflet. An arrow 62 represents a valve annulus located at the most lateral site in the mitral valve region, and an arrow 63 represents a valve leaflet tip located at the most medial site in the mitral valve region. An arrow 64 represents an anterior commissure and an arrow 65 represents a posterior commissure.

FIG. 3 illustrates an example in which the processing circuitry 144 extracts the mitral valve region as a mesh represented by a group of grid points with 19 columns and 9 rows for the anterior leaflet region and 25 columns and 9 rows for the posterior leaflet region. As illustrated in FIG. 3, when the position of each grid point is expressed as an identifier (x, y) where x denotes the number of rows and y denotes the number of columns, (8, 0) indicates the anterior commissure and (8, 18) indicates the posterior commissure. The most lateral site in the anterior leaflet and the posterior leaflet, that is, the position of x=0 is called the valve annulus, and the most medial site, that is, the position of x=8 is called the valve leaflet tip.

FIG. 3 is merely one example of the predetermined structure extracted at step S120 by the processing circuitry 144 using the acquisition function 144c, and the number, arrangement, format of the array, and the like of lattice points may be different from the example illustrated in FIG. 3. Instead of extracting the mitral valve region as a mesh represented by a group of grid points described above, the processing circuitry 144 may extract the mitral valve region as a three-dimensional image represented by a set of corresponding pixels.

FIGS. 4 to 6 illustrate examples of the predetermined structure extracted at step S120 by the processing circuitry 144 using the acquisition function 144c at each cardiac phase. FIG. 4 illustrates the mitral valve structure extracted by processing circuitry 144 using the acquisition function 144c at cardiac phase T=1 and represented in a mesh type data structure. FIGS. 5 and 6 also illustrate the mitral valve structures extracted by the processing circuitry 144 using the acquisition function 144c at cardiac phases T=2 and T=3 and represented in a mesh type data structure, respectively.

Subsequently, at step S130, the processing circuitry 144 sets, by using the setting function 144d, correction locations on the basis of the predetermined structure extracted at step S120. As an example, the processing circuitry 144 sets, by using the setting function 144d, correction locations by focusing on structural information that is invariant even though time changes in target anatomical structure, for example, after extracting the same structure at the anatomically corresponding position from the predetermined structure extracted at step S120. For example, in FIG. 4, since the length of a polyline 21, that is, the distance from a predetermined position on the annulus of a valve leaflet to a position on the valve leaflet tip is the length of the anatomical structure when the target anatomical structure is a heart valve, the distance is considered to be invariant even though time changes. That is, the length of the polyline 21 at T=1 illustrated in FIG. 4, the length of a polyline 31 at T=2 illustrated in FIG. 5, and the length of a polyline 41 at T=3 illustrated in FIG. 6 are considered to be approximately equal to one another because the polyline 21, the polyline 31, and the polyline 41 are considered to be in anatomically corresponding positions in the anatomical structure of the same subject. Accordingly, the processing circuitry 144 sets the polyline 21, the polyline 31, and the polyline 41 as correction locations by using the setting function 144d.

Similarly, the processing circuitry 144 sets, by using the setting function 144d, a polyline 22 illustrated in FIG. 4, a polyline 32 illustrated in FIG. 5, and a polyline 42 illustrated in FIG. 6, which are considered to be in anatomically corresponding positions and have the same structure, as correction locations. The processing circuitry 144 also sets, by using the setting function 144d, a polyline 23 illustrated in FIG. 4, a polyline 33 illustrated in FIG. 5, and a polyline 43 illustrated in FIG. 6, which are considered to be in anatomically corresponding positions, as correction locations. The processing circuitry 144 can specify, by using the setting function 144d, corresponding positions among a plurality of images by a process such as template matching, for example. The processing circuitry 144 determines grid points with the same identifier (x, y) in a target structure represented by a mesh, as anatomically corresponding locations, and sets correction locations.

In the above example, a case has been described in which the shape of the correction location is a polyline; however, the embodiment is not limited thereto and the shape of the correction location may be a line segment instead of a polyline. In this case, when only endpoints are designated, it is sufficient to specify the shape of the correction location. Further, the shape of the correction point may be a two-dimensional closed curve or a three-dimensional region.

In this case, the processing circuitry 144 sets, by using the setting function 144d, closed curves or the like at different times or the like in anatomically corresponding positions in the same anatomical structure of the same subject, which are considered to be invariant even though time changes in a target anatomical structure, for example, as a set of closed curves or the like to be correction locations.

The correction locations may be automatically set at step S130 by the processing circuitry 144 using the setting function 144d based on predetermined criteria, or upon receiving input of correction locations from a user through the user interface, the processing circuitry 144 may set the correction locations by using the setting function 144d.

Instead of receiving input of correction locations from a user, the processing circuitry 144 may receive input of the range of correction locations from the user by using the setting function 144d. For example, in FIG. 4, when the processing circuitry 144 receives input of a range 24 of correction locations from the user through the user interface by using the setting function 144d, the processing circuitry 144 sets, by using the setting function 144d, the pair of polylines 21, 31, and 41, the pair of polylines 22, 32, and 42, and the pair of polylines 23, 33, and 43, which are polylines included in the range 24 of correction locations, as pairs of lines to be the correction locations.

When the range 24 of correction locations received by the processing circuitry 144 from the user through the user interface by using the setting function 144d is not appropriate, the processing circuitry 144 may fail to set pairs of lines to be the correction locations within the range 24 of correction locations by using the setting function 144d. In such a case, the processing circuitry 144 may notify the user of the above situation and request the user to re-input the range 24 of correction locations, or the processing circuitry 144 may set, by using the setting function 144d, a pair of lines that are to be the correction locations and are closest to given conditions, as a pair of lines to be the correction locations. In this way, the processing circuitry 144 sets the correction locations from the same structure by using the setting function 144d.

Subsequently, at step S140, the processing circuitry 144 sets, by using the setting function 144d, an evaluation value to be used as a reference when correcting the correction locations set at step S130. As an example, the processing circuitry 144 determines, by using the setting function 144d, what feature quantities are to be used as the basis for correction in correcting the correction locations set at step S130. The processing circuitry 144 performs, by using the setting function 144d, correction using the structural information that is invariant even though time changes in the anatomical structure as the feature quantity. As an example, when the type of correction location set at step S130 is a line, the feature quantity to be used as the basis for correction is the length of the line. For example, when correcting the correction location set at step S130 by the setting function 144d, the processing circuitry 144 uses the length of the anatomical structure as a feature quantity to perform correction based on the feature quantity by using the correction function 144f. For example, the processing circuitry 144 performs, by using the setting function 144d, correction by using the distance from the predetermined position on the annulus of the valve leaflet to the position on the valve leaflet tip as the feature quantity.

As another example, the feature quantity on which the correction is based may be an angle. As an example, when correcting the correction location set at step S130 by the setting function 144d, the processing circuitry 144 uses, for example, an angle formed with a base plane or baseline obtained from a valve leaflet structure, as a feature quantity, to perform correction.

When the type of correction location set at step S130 is a closed curve, original feature quantities for correction are, for example, circumference, area, circularity, and the like. For example, when correcting the correction location set at step S130 by the setting function 144d, the processing circuitry 144 uses, for example, the area, circumference, and the like of a closed curve as feature quantities, to perform correction.

When the type of correction location set at step S130 is a three-dimensional region, the feature quantities are volume, surface area, sphericity, and the like. In this case, when correcting the correction location set at step S130 by the setting function 144d, the processing circuitry 144 uses, for example, volume, surface area, sphericity, and the like as feature quantities, to perform correction.

The setting of the feature quantities set at step S130 and serving as correction criteria may be done by the processing circuitry 144 through the user interface, manually receiving input from the user by the setting function 144d. As another example, feature quantities serving as correction criteria may be set automatically by predefined conditions. The feature quantities serving as correction criteria may be automatically determined according to the type of correction location set at step S130.

In the embodiment, a case in which step S140 is processed after step S130 is processed has been described; however, the embodiment is not limited thereto and step S130 may be processed after step S140 is processed. In this case, options incompatible with the correction criteria selected by the user at step S140 may be excluded in the process of step S130.

Subsequently, at step S150, on the basis of the correction criteria set at step S140, the processing circuitry 144 corrects the correction locations set at step S130 by using the correction function 144f.

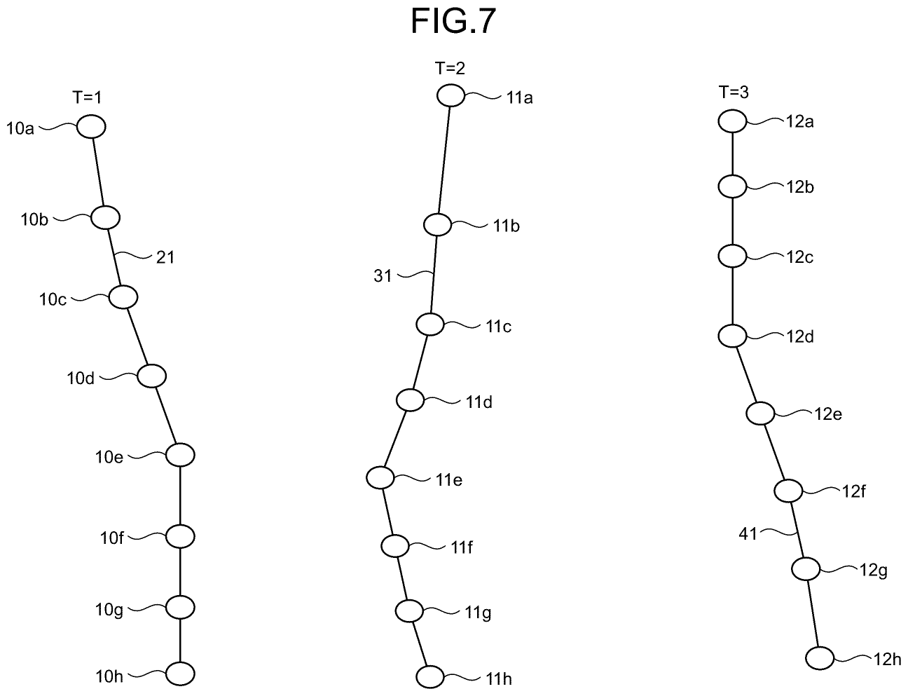

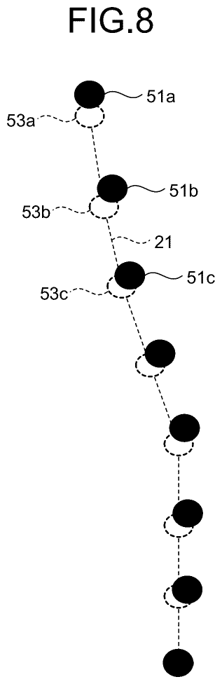

The process of step S150 is described with reference to FIGS. 7 and 8 for a case in which the type of correction location set at step S130 is a polyline and the feature quantity serving as the correction criterion set at step S140 is the length of the anatomical structure at an anatomically corresponding position. Polylines 21, 22, and 23 in FIG. 7 correspond to the polylines 21, 22, and 23 in FIGS. 4, 5, and 6, respectively. The polyline 21, the polyline 22, and the polyline 23 are data at T=1, T=2, and T=3, respectively, and these data are data corresponding to the same anatomical position.

Grid points 10a to 10h represent grid points of the polyline 21, grid points 11a to 11h represent grid points of the polyline 31, and grid points 12a to 12h represent grid points of the polyline 41. That is, the grid points 10a to 10h, 11a to 11h, and 12a to 12h correspond to points in the mesh on the polyline 21 in FIG. 4, the polyline 31 in FIG. 5, and the polyline 41 in FIG. 6, respectively.

The processing circuitry 144 calculates, by using the calculation function 144e, the feature quantities related to the form of the structure acquired from the medical image at step S110 for each of a plurality of times. As an example, the processing circuitry 144 calculates, by using the calculation function 144e, the length of the mitral valve as a feature quantity for each of the times. For example, as illustrated in FIG. 7, the processing circuitry 144 calculates, by using the calculation function 144e, the length of the polyline 21 being the length of the mitral valve at T=1, the length of the polyline 31 being the length of the mitral valve at T=2, and the length of the polyline 41 being the length of the mitral valve at T=3, as feature quantities.

As another example, the processing circuitry 144 may calculate, by using the calculation function 144e, the lengths of polylines constituting the polyline 21 and the like for each of the times, and calculate the mean, median, minimum, maximum values, and the like of the lengths of the line segments as the feature quantities.

Subsequently, the processing circuitry 144 corrects, by using the correction function 144f, the structure acquired from the medical image at step S110 so that the difference in the calculated feature quantities is reduced. As an example, the processing circuitry 144 averages, by using the calculation function 144e, the feature quantities calculated for each of the times, and corrects the structure acquired from the medical image at step S110 so that the feature quantity at each time is close to the average value of the calculated feature quantities.

FIG. 8 illustrates an example of such a process. FIG. 8 is a diagram illustrating an example of the correction process performed by the processing circuitry 144 using the correction function 144f. Grid point 53a, 53b, 53c, and the like indicate grid points before the correction process, and grid points 51a, 51b, 51c and the like indicate grid points after the correction process. That is, the processing circuitry 144 modifies, by using the correction function 144f, the positions of the grid points, for example, as indicated by the grid points 51a to 51c, so that the feature quantity at T=1 is equal to the average value of the feature quantities at the same structure at T=1, T=2 and T=3.

As an optional component, the processing circuitry 144 according to the embodiment may also perform correction by further performing an additional process. For example, with respect to grid points such as 51a, 51b, 51c, and the like in FIG. 8, the processing circuitry 144 may update, by using the correction function 144f, the positions of a plurality of grid points by further rearranging the grid points so that the distances between these grid points after being modified are equally spaced. This rearrangement of the grid points so that the distances between the grid points are equally spaced may facilitate post-processing such as the calculation of measurement values, for example.

The embodiment is not limited thereto, and the feature quantity serving as a correction criterion may be an angle, for example. As an example, when the feature quantity serving as the correction criterion set at step S140 is an angle, the processing circuitry 144 corrects, by using the correction function 144f, the grid points at each time so that an angle with a predetermined reference plane of the polyline 21, which is calculated from the corrected grid points at each time, approaches an average value of the angle with the predetermined reference plane of the polyline 21 at each time. The predetermined reference plane is, for example, the least square plane of a closed curve formed by the valve leaflet tip.

In addition, the correction locations set at step S130 may be closed curves, and the feature quantity set at step S140 and serving as the correction criterion may be circumference or area. As an example, the processing circuitry 144 corrects, by using the correction function 144f, the grid points at each time point so that a closed curve calculated from the corrected grid points at each time point approaches the average value of the closed curve at each time.

Subsequently, at step S160, the processing circuitry 144 determines, by using a determination function (not illustrated), whether the correction of all correction locations set by the setting function 144d at step S160 has been completed. When all the correction locations set at step S140 have been corrected (Yes at step S160), the correction process ends. On the other hand, when there are locations where the correction process has not been performed (No at step S160), the procedure returns to step S150, and the processing circuitry 144 performs, by using the correction function 144f, the correction process of step S150 for a pair of the correction locations where the correction process has not been completed.

The processing circuitry 144 normally performs, by using the correction function 144f, the same type of correction process for all the correction locations set at step S130; however, the embodiment is not limited thereto. The processing circuitry 144 may change, by using the correction function 144f, the process of the correction method according to the correction locations set at step S130. As an example, the processing circuitry 144 may change, by using the correction function 144f, the method for the correction process performed at step S160 according to the position of the correction location.

The embodiment is not limited to a case in which the correction process is performed for all the correction target locations, but the correction process may be performed only for some of the correction locations set at step S130. As an example, the processing circuitry 144 may further perform a process of determining whether to perform the correction process for each pair of correction locations at step S160. As an example, when the processing circuitry 144 first calculates, by using the calculation function 144e, the correction criteria at step S140 with respect to a pair of correction locations set at step S130 and the difference in the correction criteria for the pair of correction locations is equal to or less than a threshold value, the processing circuitry 144 may perform no correction process for the correction locations at step S140. The threshold value may be set in advance, or may be set by receiving user input from the user interface. As another example, at step S130 or step S140, the processing circuitry 144 may calculate the difference in the correction criteria by using the correction function 144f, and it is possible not to set pairs whose difference of the correction criteria is below the threshold value as correction locations at step S150.

As described above, in the first embodiment, the processing circuitry 144 calculates feature quantities related to the form of the same structure acquired from a plurality of medical images captured at different times or by different devices, for each of the medical images, and corrects the structure so that the difference in the feature quantities is reduced. The processing circuitry 144 performs a correction process so that corresponding feature quantities, for example, anatomical structure lengths, are matched at anatomically corresponding positions at different times, for example. This allows the correction process of time-series images to be performed while keeping anatomical feature quantities in a reasonable form.

### First Variation of First Embodiment

The first embodiment has described a case in which the processing circuitry 144 sets lines or planes that divide a structure acquired from a medical image into a plurality of regions by using the setting function 144d, calculates feature quantities related to the form of the structure for each of the regions by using the calculation function 144e, and corrects the structure by using the correction function 144f so that the difference in the feature quantities is reduced. However, the embodiment is not limited thereto, and the processing circuitry 144 may calculate an evaluation value on the basis of the form and shape of the structure by using the calculation function 144e, and select a method of correcting the structure on the basis of the calculated evaluation value, by using the correction function 144f.

The form and shape here is, for example, a curvature. That is, the processing circuitry 144 may calculate, by using the correction function 144f, the curvature as an evaluation value for each of the divided regions, and select a method of correcting the structure on the basis of the calculated evaluation value. As an example, when the curvature is large, the reliability of the process by which the processing circuitry 144 acquires the structure by using the acquisition function 144c at step S120 may be low. In this case, at step S150, for example, the processing circuitry 144 corrects, by using the correction function 144f, a structure with a higher curvature to match a structure with a lower curvature among the pairs set for the correction locations at step S130. That is, the processing circuitry 144 selects, by using the correction function 144f, a method of correcting a first correction location in accordance with a second correction location when the curvature of the first correction location is greater than the curvature of the second correction location among the pairs set for the correction locations at step S140, and selects a method of correcting the second correction location in accordance with the first correction location when the curvature of the first correction location is smaller than the curvature of the second correction location. In other words, the processing circuitry 144 selects, by using the correction function 144f, a method of correcting the structure according to the reliability of the process of acquiring the structure.

As another example, the processing circuitry 144 may ignore, by using the correction function 144f, data at times when the curvature of the correction location is high and perform correction based on data at times when the curvature of the correction location is small.

As another example, when the difference in shape between correction locations in the same pair is large compared to another pair set for correction locations at step S130, since the reliability of the process of acquiring the structure at step S120 in that pair may be low, the processing circuitry 144 may correct, by using the correction function 144f, correction locations of a pair with a large difference in shape between the correction locations in accordance with correction locations of a pair with a small difference in shape between the correction locations.

As another example, when performing the process of step S120 for each of a plurality of times, the processing circuitry 144 may calculate, by using the calculation function 144e, the likelihood of the structure acquired from the medical image for each pixel, that is, the probability of the pixel being the structure, as an evaluation value for each pixel, and select, by using the correction function 144f, a method of correcting the structure on the basis of the calculated evaluation value. That is, the evaluation value calculated by the processing circuitry 144 using the calculation function 144e is the likelihood of the structure. Here, the processing circuitry 144 calculates the likelihood using, for example, U-Net, by using the calculation function 144e. A correction location including more pixels with high likelihood among the pairs set for the correction locations is considered to be more reliable than another correction location including more pixels with low likelihood. Accordingly, the processing circuitry 144 corrects, by using the correction function 144f, the other correction location in accordance with the correction location including more pixels with high likelihood.

As described above, in the first variation of the first embodiment, the processing circuitry 144 calculates an evaluation value and the like, and further changes a method for the correction process based on the calculated evaluation value and the like. This further improves the accuracy of the correction process.

### Second Variation of First Embodiment

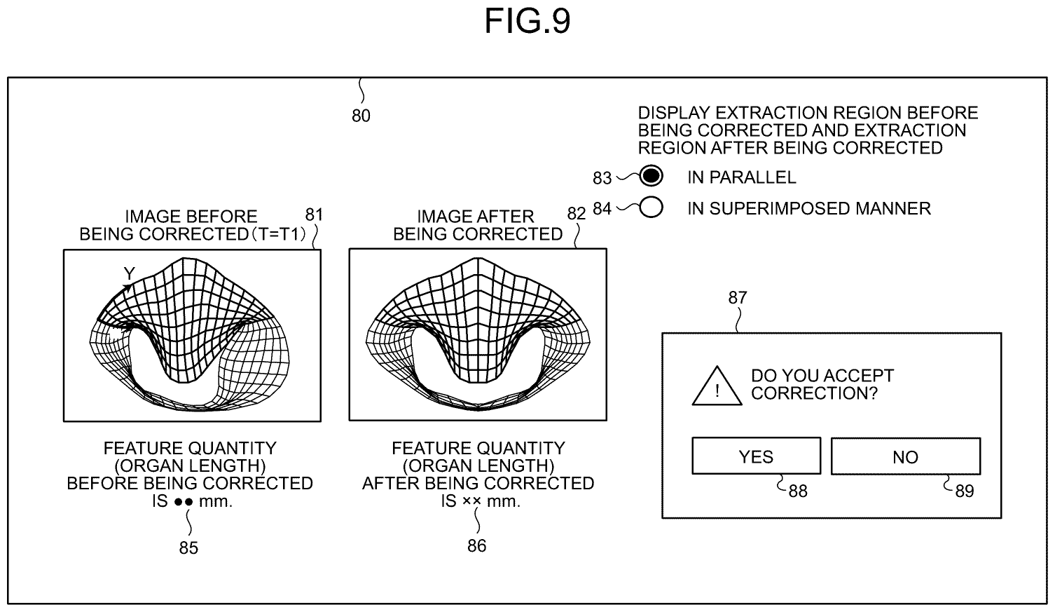

The second variation of the first embodiment describes a user interface that displays a corrected image to a user. FIG. 9 illustrates an example of such a user interface. When correction of all correction locations is completed at step S160, the processing circuitry 144 causes a display screen 80 of the display 142 to display an image 82 of a region including corrected correction locations by using the control function 144a. Upon receiving an instruction from a user through a button 83 to display an image before being corrected and an image after being corrected in parallel by using the control function 144a, the processing circuitry 144 causes the display 142 to display the image 82 after being corrected together with an image 81 before being corrected by using the control function 144a. Upon receiving an instruction from the user through a button 84 to display an image before being corrected and an image after being corrected in a superimposed manner by using the control function 144a, the processing circuitry 144 causes the display 142 to display the image before being corrected and the image after being corrected in a superimposed manner by using the control function 144a. That is, the processing circuitry 144 causes the display unit to display a medical image before being corrected and a medical image after being corrected in a superimposed manner by using the control function 144a.

The processing circuitry 144 causes the display 142 to display a screen 87 to ask the user, who has referred to the image 82 after being corrected, whether to accept the correction by using the control function 144a. When the processing circuitry 144 receives, by using the control function 144a, a user's selection to accept the correction through a button 88, the processing circuitry 144 receives the image 82 after being corrected as a correctly corrected medical image and stores the image 82 after being corrected in the memory 141. On the other hand, when the processing circuitry 144 receives, by using the control function 144a, a user's selection not to accept the correction through a button 89, the processing circuitry 144 discards the image 82 after being corrected and either terminates the process or performs the correction process by changing the conditions. In this case, the processing circuitry 144 may receive input of the changed conditions from the user by using the control function 144a.

As described above, in the second variation of the first embodiment, the processing circuitry 144 includes a user interface for performing a process such as displaying a corrected image to a user or receiving input from a user. This improves usability.

### Third Variation of First Embodiment

The embodiment is not limited to the above examples. As an example, at step S160, the processing circuitry 144 may calculate, by using the calculation function 144e, feature quantities or measurement values with respect to corrected correction locations, and display measurement values before being corrected and measurement values after being corrected to a user. As an example, as illustrated in FIG. 9, the processing circuitry 144 causes the display 142 to display the measurement values before being corrected as a message 85 directed to a user and to display the measurement values after being corrected on the display 142 as a message 86 directed to the user by using the control function 144a.

The processing circuitry 144 also causes the display 142 to display the screen 87 to ask the user, who has referred to the message 86 directed to the user and related to the measurement values after being corrected, whether to accept the correction by using the control function 144a. When the processing circuitry 144 receives, by using the control function 144a, a user's selection to accept the correction through the button 88, the processing circuitry 144 receives the image 82 after being corrected as a correctly corrected medical image and stores the image 82 after being corrected in the memory 141. On the other hand, when the processing circuitry 144 receives, by using the control function 144a, a user's selection not to accept the correction through the button 89, the processing circuitry 144 discards the image 82 after being corrected and either terminates the process or performs the correction process by changing the conditions. In this case, the processing circuitry 144 may receive input of the changed conditions from the user by using the control function 144a.

As described above, in the third variation of the first embodiment, the processing circuitry 144 further displays measurement values for corrected correction locations to a user. This improves usability.

### Fourth Variation of First Embodiment

In the previous embodiments, a case in which a target anatomical structure is the mitral valve has been described; however, the embodiment is not limited thereto. In the embodiment, target organs may include organs other than the heart, such as the brain, vocal cords, and uterus. Since these organs have feature quantities that are invariant over time, the processing circuitry 144 corrects the structure by using the correction function 144f so that the difference in feature quantities is reduced. This allows the processing circuitry 144 to perform the same correction process for organs other than the heart.

According to at least one of the embodiments described above, image quality can be improved.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An image information processing apparatus comprising:
processing circuitry configured
to acquire a same structure from a plurality of medical images captured at different times or by different devices,
to calculate feature quantities related to a form of the structure for each of the medical images, and
to correct the structure so that a difference in the feature quantities is reduced.

2. The image information processing apparatus according to claim 1, wherein the processing circuitry is configured to acquire shape information at anatomically corresponding positions of anatomical structure of a same object included in the medical images, as the structure.

3. The image information processing apparatus according to claim 2, wherein the feature quantity is a length of the anatomical structure.

4. The image information processing apparatus according to claim 2, wherein the feature quantity is structural information that is invariant even though time changes in the anatomical structure.

5. The image information processing apparatus according to claim 2, wherein
the anatomical structure is a heart valve, and
the feature quantity is a distance from a predetermined position on an annulus of a valve leaflet to a position on a valve leaflet tip.

6. The image information processing apparatus according to claim 1, wherein
the processing circuitry is configured to
calculate an evaluation value on a basis of a form of the structure, and
select a method of correcting the structure on a basis of the calculated evaluation value.

7. The image information processing apparatus according to claim 1, wherein
the processing circuitry is configured to
set correction locations from the same structure on, and
calculate the feature quantities for the set correction locations.

8. The image information processing apparatus according to claim 1, wherein the processing circuitry is configured to correct the structure by using a length, an angle, a circumference, or an area as a correction criterion.

9. The image information processing apparatus according to claim 1, wherein the processing circuitry is configured to select a method of correcting the structure according to a reliability of a process of acquiring the structure.

10. The image information processing apparatus according to claim 1, wherein the processing circuitry causes a display unit to display the medical image before correction is performed and a medical image after correction is performed in a superimposed manner.

11. The image information processing apparatus according to claim 6, wherein the evaluation value is a likelihood of the structure.

12. The image information processing apparatus according to claim 11, wherein the processing circuitry is configured to correct another correction location in accordance with a correction location including more pixels with high likelihood.

13. An image information processing method comprising:
acquiring a same structure from a plurality of medical images captured at different times or by different devices;
calculating feature quantities related to a form of the structure for each of the medical images; and
correcting the structure so that a difference in the feature quantities is reduced.

14. A non-transitory computer readable medium including programmed instructions, wherein the instructions, when executed by a computer, cause the computer to perform:
acquiring a same structure from a plurality of medical images captured at different times or by different devices;
calculating feature quantities related to a form of the structure for each of the medical images; and
correcting the structure so that a difference in the feature quantities is reduced.
